(19) United States
(12) Patent Application Publication
(10) Pub. No.: US 2010/0023016 A1
(43) Pub. Date: Jan. 28, 2010

(54) TOOLS AND METHODS FOR ORTHOPEDIC SURGERY

(76) Inventor: GARY BOTIMER, NAMPA, ID (US)

Correspondence Address: PEDERSEN & COMPANY, PLLC, P.O. BOX 2666, BOISE, ID, 83701, US

(21) Appl. No.: 12/509,438

(22) Filed: Jul. 24, 2009

Related U.S. Application Data
(60) Provisional application No. 61/083,460, filed on Jul. 24, 2008.
(60) Provisional application No. 61/083,514, filed on Jul. 24, 2008.
(60) Provisional application No. 61/092,837, filed on Aug. 29, 2008.
(60) Provisional application No. 61/121,795, filed on Dec. 11, 2008.
(60) Provisional application No. 61/155,148, filed on Feb. 24, 2009.
(60) Provisional application No. 61/121,097, filed on Dec. 9, 2008.

(51) Int. Cl. A61F 5/00

(52) U.S. Cl. 606/87

(57) ABSTRACT

Tools may be used individually and/or in combination to allow minimally invasive and safer orthopedic surgery. A femur adjustment tool lifts and lateralizes the proximal end of the femur during hip replacement surgery by pivoting on a ball temporarily placed in the acetabulum. A tissue protection and broach (rasp, cutting, drilling) guide tool may retract tissue at the incision, protect tendons and soft tissue, and provides a curved, elongated surface that cradles and guides the broaching tool. A tip of the main body of the protection and guide tool, and a hook protruding from the main body, may extend along opposite surfaces of the femur to help “capture” a portion of the femur for tool stability and to effectively and positively protect the piriformus tendon that will reside in the “V” between the hook and tip. A bone clamp is used when a generally transverse cut is made across a bone, for example, a knee surgery proximal tibial cut, wherein the clamp improves control of the bone portion for safer freeing of the bone portion from soft tissue and extracting the bone portion from the incision. A broad, flat plate of the bone clamp may be slid between the bone and the bone portion into the narrow space that has been created by cutting the bone, and a relatively narrow gripping member may be slid along/across the opposing surface of the bone portion, which allows the narrow gripping member to fit into the intercondylar notch of the femur.

This application claims benefit of Provisional Application 61/083,460, filed Jul. 24, 2008; Provisional Application 61/083,514, filed Jul. 24, 2008; Provisional Application 61/092,837, filed Aug. 29, 2008; and Provisional Application 61/121,795, filed Dec. 11, 2008; and Provisional Application 61/155,148, filed Feb. 24, 2009; wherein the entire disclosures of which are incorporated herein by this reference.

### BACKGROUND OF THE INVENTION

1. Field of the Invention

This invention relates generally to tools and methods for orthopedic surgery. In one group of embodiments, the tool and methods are specially-adapted for hip replacement or other hip surgery. In another group of embodiments, the tool and methods are specially-adapted for knee replacement and other knee surgery. More specifically, the invention is a system, and individual tools, for improved hip and/or knee arthroplasty procedures and results. One of the tools is used to conveniently move/lift the femur of the patient relative to the acetabular socket and the surgical incision, and to hold the femur so that the required work at or near the top end of the femur may be efficiently done. Another tool is a combination tool that may be used for tissue protection, broach guiding, and, optionally, for femur-lift assistance. Another tool is a bone clamp that may be used to grasp, manipulate, and free portions of a bone, for example, a portion of the tibia during knee surgery. Using one or multiple of these tools improves the efficiency and effectiveness of joint arthroplasty, especially in terms of reduced invasiveness and reduced tissue damage.

2. Related Art

In the field of hip replacement surgery, it is often necessary to separate the top (proximal end) of the femur from the hip acetabular socket. This way, the top of the femur may be worked on to prepare it for being properly received by the acetabulum or acetabulum prosthesis component. For example, a new “ball” surface at the top of the femur may be provided during the surgery, for being better received by a new inner acetabulum surface also provided during the surgery, in order to repair/restore the hip “ball and socket” joint.

Typically, during the surgery, after the top of the femur has been removed from the hip socket, it must be backed away from or otherwise distanced from the hip socket and moved toward/out of the surgical incision in order for the femur end (top/proximal end) to be conveniently accessed and worked on by the surgeon. This work by the surgeon includes “broaching” of the femur, which comprises the use of a rasp to drill axially (longitudinally) into the femur to create an axial hole for insertion and anchoring of a femur prosthesis. The prosthesis will typically be a shaft, inserted and anchored into the drilled hole, that has an artificial hip joint ball at its end.

Moving of the femur for better access to the surgeon for this “broaching” typically takes the form of “lateralizing” and “lifting” the femur end, to move it up and out of, or substantially out of, the surgical incision. Hand-held retractors (R) may be used to hold back the soft tissue around the incision, as shown in FIGS. 7-10, to further expose the top end of the femur. Conventionally, this “lateralization” and “lifting” has been done by hand by one or more surgical assistants, by manipulating the patient's leg to push the femur top toward the incision opening, therefore requiring additional, trained surgery room personnel and resulting in a difficult and/or clumsy procedure.

Conventional hand-held tissue retractors, such as are illustrated as R1 and R2 in Provisional Application 61/155,148, incorporated herein, are typically used in hip arthroplasty, for retracting tissue around the incision. One of the retractors R1 may be approximately parallel with the length of the patient's leg, with the flat surface of the retractor (R1) at an angle to the plane of the incision. The surgeon may use the flat surface of such a retractor as a rough guide for the broach, by generally aligning the length of the broach with the length of the retractor and then guiding the broach generally parallel to the retractor to come near to the femur. However, the surgeon must soon “turn the corner” with the broach to drill into the length of the femur, rather than drill down past the femur into the surrounding thigh tissue. The area of the incision and the top end of the femur includes much soft tissue, including skin, muscle, and tendons, which can be damaged if the broach slips off of the flat surface of the conventional retractor or the broach moves downward into the space inside the thigh (between the inward end of the conventional retractor and the femur) that contains muscle, tendons, and other soft tissue that are easily damaged by the broach.

There is a need, then, to conveniently and efficiently “lateralize and lift” the top of the femur during hip surgery, in a consistent and predictable manner and without surgeon's assistants having to manually manipulate the leg of the patient in the conventional manner. There is a need, also, to improve the method of broaching and minimize or eliminated damage of surrounding tissue.

In the field of knee arthroplasty, or other surgery that requires cutting and removal of a portion of a bone, there is a need for an improved hand-held tool for clamping and/or grasping bone. There is a need, in the inventor's opinion, for a tool to be used in procedures that require a transverse cut through a bone, for example, a proximal tibial cut during knee reconstruction. The inventor believes that there is a need for a clamping tool for manipulating a bone portion that has been cut from the tibia, wherein the tool allows improvements in the steps of removing soft tissue from the bone portion and removing the bone portion from the patient's body, and because of the improvement in these steps, may allow the original proximal tibial cut to be made in a less aggressive manner.

The invented tools, individually and/or in combination, address the above-discussed needs and/or other needs in orthopedic surgery.

### SUMMARY OF THE INVENTION

The present invention comprises one or more tools for use in orthopedic surgery, and/or methods of using said one or more tools in orthopedic surgery or other procedures. A first preferred tool is a femur adjustment tool for use in hip replacement or other hip surgery. A second preferred tool is a tissue protection and guide tool for a broach or other cutting/drilling tool, which second tool may also be used to retract/control soft tissue and/or to assist in lifting or positioning the femur. A third preferred tool is a bone clamp device that may be used to grasp, manipulate, and free portions of a bone.

The femur adjustment tool “lateralizes and lifts” the top end of the patient's femur into, and preferably holds the femur in, the desired position so that the femur end may be conveniently accessed and effectively worked on during the surgery. The femur adjustment tool of the present invention comprises a fulcrum member that is received against/in the acetabulum, and a lever unit connected to said fulcrum member. The femur adjustment tool acts as a first-class lever system for lifting and lateralizing the femur, wherein the preferred fulcrum member is located between the input effort and the output load. In operation, the handle end is pushed, which causes the lever unit to swing about the fulcrum that is located in/on the acetabulum, overcoming the resistance force on the femur side, which is the femur's resistance to movement due to it being in a location inside the patient's body that is normal/natural except that it has been separated from the hip socket. The fulcrum is nearer the femur contact end than the handle end, to provide the benefit of leverage. The fulcrum member preferably comprises a ball-like, spherical, partly spherical, or other rounded or curved (and preferably smooth) portion that contacts and pivots on the acetabulum, so that the acetabulum is not damaged or marred by the fulcrum member during use of the femur adjustment tool.

The preferred tissue protector and broach guide tool may serve multiple functions during surgery, preferably including shielding tissue from possible damage during broaching or other bone cutting/drilling procedures and guiding the broaching or other cutting/drilling tool for effective, accurate, and safe broaching/cutting/drilling. In addition, the preferred protector and guide tool may serve as a soft tissue retractor to assist in control of the soft tissue around or inside the surgical incision. The preferred tissue protector and broach guide tool comprises an elongated, preferably rigid arm that bends or curved at approximately 20-45 degrees, to form a handle portion and a guide portion. The handle portion is adapted for being held and/or forced by a user for placing a tip of the guide portion against a surface of the femur, so that a curved/concave surface of the guide portion is generally aligned with, and reaches to, the femur portion into which the broaching tool will be forced to create a hole for implantation/connection of the ball-portion of a prosthetic hip joint. An extension member may protrude from the guide portion to curve/bend around to a posterior region of the femur, to further stabilized the protector and guide tool relative to the femur. The tool is preferably adapted, for example, by a space between the guide portion and the extension member, to allow space for, and to protect, the piriformis tendon, which is near the femur portion to be broached and that is therefore vulnerable to damage during conventional hip arthroplasty procedures. The guide portion will shield the piriformis tendon from the broaching tool and, due to its curved/concave shape, will prevent the broaching tool from sliding or falling toward the piriformis tendon and, hence, will prevent damage to said tendon.

While one or the other of the femur adjustment tool and protector and guide tool may be used individually in surgery, a combination of both tools has been found by the inventor to be especially beneficial, especially in hip arthroplasty. The femur adjustment tool may be used to lift and lateralize the femur, and the protector and guide may be used at generally the same time to protect tendon(s) and other soft tissue while guiding the broaching tool accurately toward and into the femur. This preferred combination of tools is particularly effective in reducing invasiveness, uncertainty, and damage during hip arthroplasty.

The preferred bone clamp tool may be used in multiple procedures that require or may be enhanced by clamping and/or grasping bone. The bone clamp tool is used in procedures that require a cut through a bone wherein the cut-off/separated bone portion must be freed from soft tissue attached to the bone portion and/or withdrawn from a relatively small incision. The bone clamp is particularly effective when a transverse cut is made through a bone, for example, a proximal tibial cut during knee reconstruction. The bone clamp tool is used to clamp and manipulate a bone portion that has been cut from the tibia, wherein the tool allows improvements in the steps of removing soft tissue from the bone portion and removing the bone portion from the patient's body, and because of the improvement in these steps, may allow the original proximal tibial cut to be made in a less aggressive, and therefore safer and less invasive, manner.

### BRIEF DESCRIPTION OF THE DRAWINGS

FIG. 1 is a first side perspective view of one embodiment of the invented femur adjustment tool.

FIG. 2 is a second, opposite side view of the embodiment of FIG. 1.

FIG. 3 is a front view of the embodiment of FIGS. 1 and 2.

FIG. 4A is a first side perspective view of another embodiment of the invented femur adjustment tool.

FIG. 4B is a second, opposite side view of the embodiment of FIG. 4A.

FIG. 4C is a front view of the embodiment of FIGS. 4A and B.

FIG. 5A is a rear side view of an alternative endplate and ball connection anchor piece that may be used in embodiments of the femur adjustment tool (without ball connected).

FIG. 5B is a side view of the endplate and ball connection anchor piece of the embodiment of FIG. 5A (without ball connected).

FIG. 5C is a rear view of the embodiment of FIGS. 5A and B, with a ball connected to one of the anchor locations.

FIG. 5D is a rear view of the embodiment of FIGS. 5A-C, with a ball connected to the other one of the anchor locations.

FIG. 6A is a rear side view of an alternative endplate and ball connection system that may be used in embodiments of the femur adjustment tool (with ball connected and swiveled and locked in a position wherein the ball extends to the right in the figure).

FIG. 6B is a rear side view of the embodiment of FIG. 6A (with ball connected and swiveled and locked in a position wherein the ball extends to the left in the figure).

FIG. 7 is a side perspective view of one embodiment of the invented tissue protector and guide tool that may be used by itself in orthopedic surgery or with an embodiment of the invented femur adjustment tool.

FIG. 8 is a top view of the embodiment of FIG. 7.

FIG. 9 is a side view of the embodiment of FIGS. 7 and 8.

FIG. 10 is a rear view of the embodiment of FIGS. 7-9.

FIG. 11 is a side view of an alternative embodiment of a tissue protector and guiding tool, which is similar to that in FIGS. 7-9, but which has a greater obtuse angle between the handle portion/end and the protector portion/end of the tool.

FIG. 12 is a view of the embodiment of FIG. 11, viewed along the line 12-12 in FIG. 11.

FIG. 13A is a perspective view of an alternative endplate and ball connection system for a femur adjustment tool according to embodiments of the invention, showing options for connection of balls at two locations on the endplate.

FIG. 13B is a perspective view of the endplate and ball connection system of FIG. 13A, with a ball secured and fixed at one of the two locations.

FIGS. 14A and B are schematic side and top views of the embodiment of FIGS. 13A and B, illustrating preferred angles of the ball/shaft relative to the plane of the endplate.

FIGS. 15 and 16 are side illustrations of preferred steps of using embodiments of the femur adjustment tool to lift and lateralize a femur neck. The pelvic bone, with its acetabulum, and the femur bone are shown in cross-section.

FIG. 17 is a perspective illustration of one method of using an embodiment of the invented tissue protector and guiding tool (TPG tool).

FIG. 18 is another illustration of the method of FIG. 17, wherein a broach is being impacted into the femur by using the TPG tool, with the femur shown in cross-section.

FIG. 19 is an illustration of one method of using, in combination, an embodiment of the invented femur adjustment tool and one embodiment of the invented TPG tool for hip arthroplasty.

FIG. 20 is a side view of one embodiment of an invented bone clamp, with a ratchet-style latch.

FIG. 21 is a top view of the bone clamp of FIG. 20.

FIG. 22 is a rear view of the bone clamp of FIGS. 20 and 21.

FIGS. 23-26 are anterior views of some, but not the only, methods of using the bone clamp of FIGS. 20-22. These figures show sequential steps of cutting a femur, clamping the bone portion, and removing the bone portion.

FIGS. 27-29, and 31 are lateral views of generally the same steps as are shown in FIGS. 23-26. FIG. 30 is a top view of the clamp above the surface of the tibia, illustrating the preferred size and shape of the base-plate compared to the cut surface of the tibia.

### DETAILED DESCRIPTION OF THE PREFERRED EMBODIMENTS

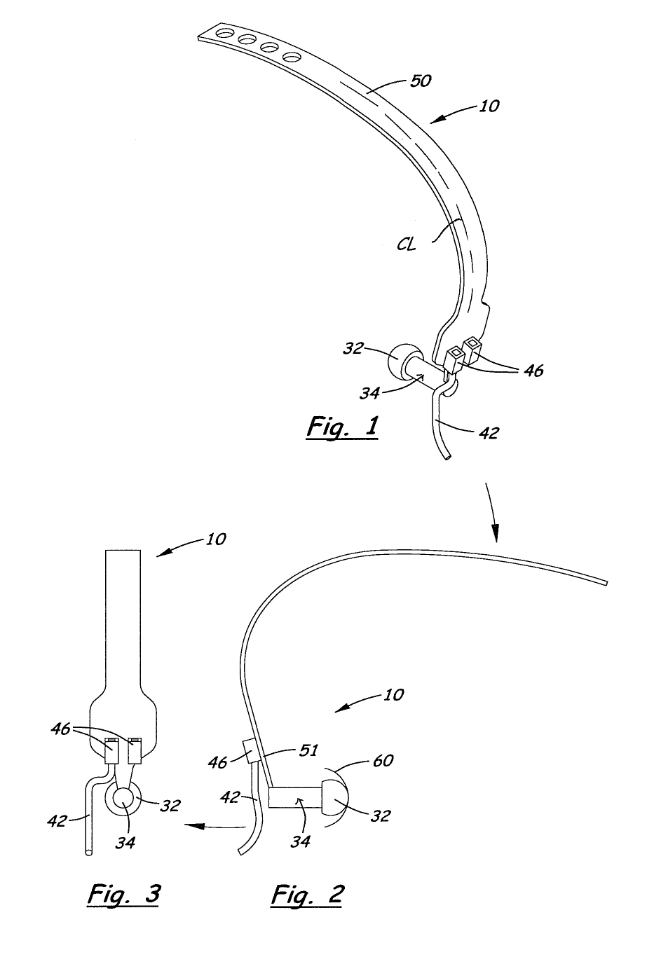

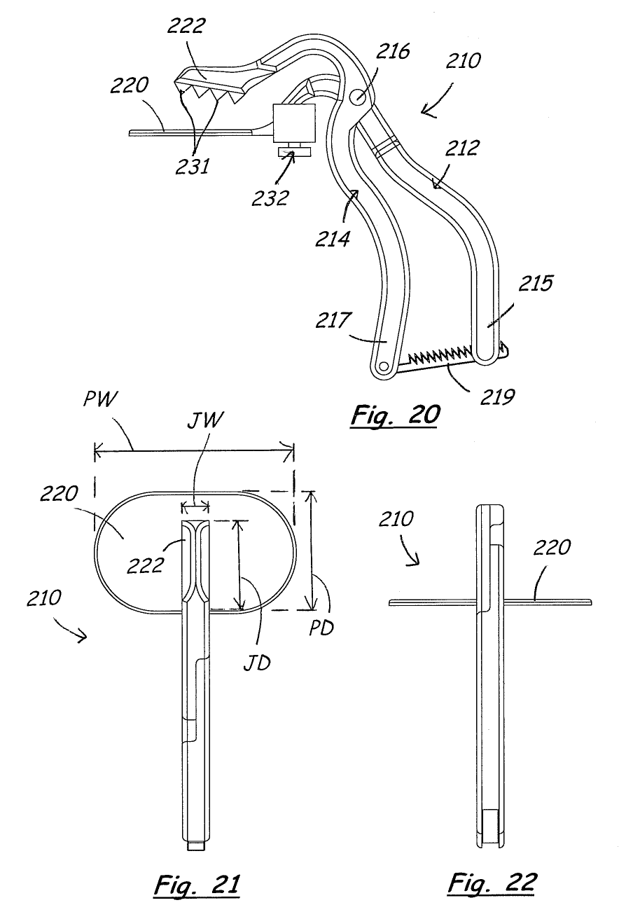

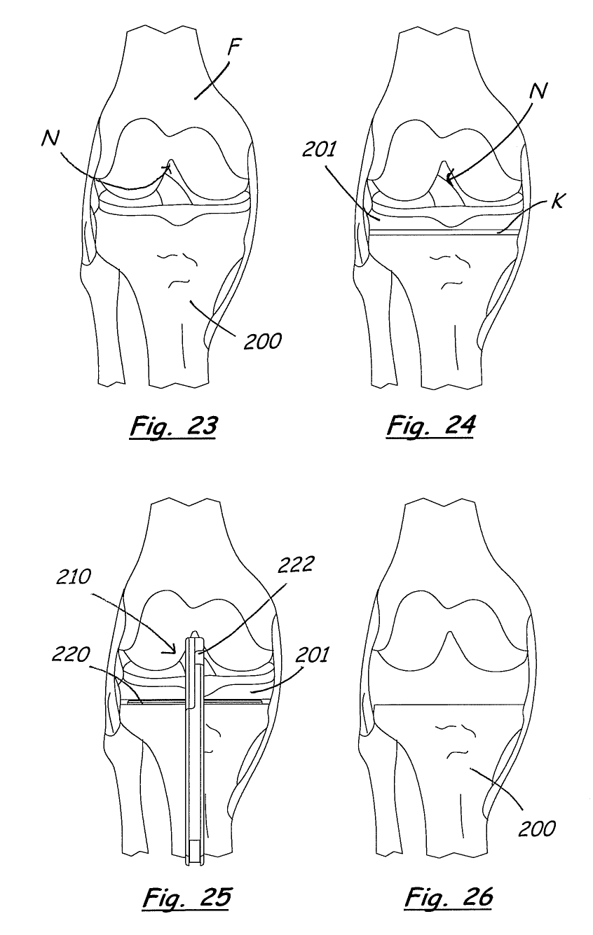

Referring to the Figures, there are depicted several, but not the only, embodiments of the present invention. FIGS. 1-3, 4A-C, 5A-D, 6A and B, 13A and B, 14A and B, 15, 16 and 19 portray preferred embodiments and use of the femur adjustment tool. FIGS. 7-12, and 17-19 portray preferred embodiments and use of tissue protector and guide tool. FIGS. 20-31 portray preferred embodiments and use of the invented bone clamp. Either one, two or three of the invented tools may be used to improve arthroplasty, especially hip and knee arthroplasty. The three preferred tools, and methods of using them, are discussed in detail below, and it should be understood that the individual tools are not necessarily limited to the embodiments shown and detailed herein, and that, while the preferred methods are described herein, that other uses for the tools, individually or various combinations of two or more, are included within the broad scope of the invention.

### Femur Adjustment Tool

The preferred femur adjustment tool “lateralizes and lifts” the top end of the patient's femur into, and preferably holds the femur in, the desired position so that the femur end may be conveniently accessed and effectively worked on during the surgery. This “lateralize and lift” motion provided by the placement and shape of the preferred femur adjustment tool helps makes the broaching of the proximal femur easier and safer in preparation for the subsequent steps in the hip replacement surgery.

The femur adjustment tool of the present invention comprises a ball or other fulcrum member that is received against/in the acetabulum, and a lever unit connected to said fulcrum member. According to the instant inventor, there are no current femoral extractors known or available that use a ball or other fulcrum member/point in the acetabulum as a pivot point for moving the proximal femur with a rod or other femoral force member connected to the same tool. The lever unit of the femur adjustment tool has a handle end and a femoral contact end, with the fulcrum member being between these ends, so that the femur adjustment tool acts as a first-class lever system for lifting and lateralizing the femur. Thus, the preferred fulcrum member is located between the input effort (surgeon, surgeon's assistant, and/or a mechanized system for applying force) and the output load (moving and/or supporting the femur). In operation, the handle end is pushed, which causes the lever unit to swing about the fulcrum that is located in/on the acetabulum, overcoming the resistance force on the femur side, which is the femur's resistance to movement due to it being in a location inside the patient's body that is normal/natural except that it has been separated from the hip socket. The fulcrum is nearer the femur contact end than the handle end, to provide the benefit of leverage.

In preferred embodiments of the femur adjustment tool, the handle end is formed by an elongated band or other elongated lever bar. In preferred embodiments of the femur adjustment tool, the femoral contact end (also called a “femoral component”) comprises at least one, preferably curved, arm that is adapted to extend along a posterior portion of the femur to contact one or more surfaces of the femur. The elongated lever bar, fulcrum member, and at least one arm are arranged relative to each other so that force upon the lever bar will cause the device to pivot on the acetabulum surface so that the femoral arm(s) lateralize(s) and lift(s) the femur into the desired position. To accomplish this, the connections between the fulcrum member, the lever bar, and the femoral arm(s) are preferably rigid and fixed during use in surgery, so that there is no relative movement between these three components during use on the patient. However, one or more of said rigid and fixed connections may be changeable, moveable, or otherwise adjustable and then immovably fixable prior to surgery, for example, so that the femur adjustment tool may be adapted to each patient's particular anatomy, for example, the dimensions and structure of the particular patient's femur and hip joint, or for a left femur or a right femur arthroplasty.

The invented femur adjustment tool may be a hand tool, wherein the user grasps and applies force to the lever bar to pivot the tool, and hence the femur, into the desired position, and then manually holds and retains the femur adjustment tool in that position during the surgeon's work on the femur. Or, the invented femur adjustment tool may be secured to a support, holder, arm or other mechanism (hereafter, support/holder) at some time during the procedure, for example, to hold the femur adjustment tool prior to, during, and/or after the desired pivoting force has been applied. Therefore, the desired pivoting force may be applied and maintained solely by a person (for example, surgeon, by a surgeon's assistant, or other personnel), by said person with assistance from said support/holder, or substantially or entirely by said support/holder.

The handle/lever bar of the preferred femur adjustment tool (the proximal end of the tool, as defined by being the end typically manipulated by an assistant's hand), preferably has a gently arching geometry so that the tool extends out from the user's hand and/or the tableside support/holder, arches over the body of the patient on the operating table, and extends over and above the surgical incision where the hip is being operated on. This “extending out and over” geometry of the femur adjustment tool may be provided by a single curved band, but may, of course, be obtained by structures other than the single curved band.

The handle/lever bar may have holes, slots, or ridges or other means (hereafter holes/slots) for aiding in the user grasping and applying force. Alternatively or additionally, said holes/slots may cooperate with said support/holder for adjustably securing the femur adjustment tool into a support/holder near on or near the surgical table, for example, by receiving a bolt or screw on the support/holder. Or, the series of holes/slots may receive a clip or spring-biased ball or rod on the support/holder. In any event, the handle/lever bar of the instant femur adjustment tool may be adapted to be held manually by a human user, and/or adjustably and securely connected to a support/holder near the side of the surgery table, for example. If connected to a support/holder, the femur adjustment tool and support/holder combination is preferably adapted for convenient and secure placement of the tool in proper location, yet easy and quick movement of the tool and support/holder out of the way when it the tool is no longer needed. In a case wherein the desired pivoting force is applied and/or maintained substantially or entirely by said support/holder, as mentioned above, it will be understood that additional mechanisms and/or programming may be needed for effective operation of said support/holder, for example, a robotic arm and/or other controllable system.

Due to the close proximity of the fulcrum member to the femur contact end/ femur arm, the combination of fulcrum member and femur arm may be said to be the distal end of the femur adjustment tool, wherein “distal” is defined as the end in/near the incision and by being opposite the proximal end held by the user and/or the support/holder. The fulcrum member may also be called the “acetabular component,” because it cooperates and interacts with the socket of the hip joint, and is a ball-like structure that fits in the hip ball socket. Preferably, the ball structure is a “standard trial head” ball provided by orthopedic surgery supply companies, so that various sizes of trial head balls may be provided and removably and adjustably connected to the femur adjustment tool, to become the fulcrum member, as is appropriate for the size and shape of the patient's socket. This way, one part of the distal end of the femur adjustment tool has a “ball” structure for engaging the socket of the patient's hip joint being worked on, while the proximal end of the femur adjustment tool is securely held in the user's hand and/or engaged in the table-side holder/support. The tool placement, therefore, comprises one part of the distal end of the femur adjustment tool being in contact with and preferably firmly engaging, the hip joint's socket so that force applied to the proximal end (handle/lever bar) will cause the femur adjustment tool to pivot against the hip socket, in a way that tends to be safe and predictable.

The femoral arm or “femoral component” of the preferred femur adjustment tool, which interacts with and engages the top end of the femur, is preferably a single “rod” structure that is of a small-enough diameter to fit under (posterior relative to) the femur, to assist in lifting the femur up and toward/out of the surgical incision, and as long as the force is maintained by the user and/or a support/holder, the rod structure securely supports the femur there, in place for further work. This way, with the first section of the distal end of the subject femur adjustment tool firmly engaged in the hip socket, the second section of the distal end of the tool is able to firmly engage a surface of the separated femur, and upon manipulation by the surgical assistant or other personnel or the support/handle of the handle portion of the femur adjustment tool, the femur is “lateralized and lifted” into an easily-accessible position and firmly held there for further work.

To better fit the patient and optimize leverage application, the ball structure or other fulcrum member may be distanced from the lever unit, and/or moved or pivoted to alternative positions on the lever unit, and/or moved or pivoted to alternative angles relative to the lever unit, and/or other portions of the femur adjustment tool may be adjusted relative to each other, preferably prior to use of the tool on the patient. For example, the femur arm/rod may be lengthened relative to, and/or pivoted to alternative angles, relative to the fulcrum member and/or lever bar. Also, for example, the ball structure may be rigidly and immovably attached to the lever unit at different locations, and/or be pivotal/moveable and lockable in place for femur adjustment based on the patient's body structure and/or based on whether the surgeon is operating on a left or right hip. This may be done, for example, by providing multiple attachment points for the ball structure on the lever unit, and/or by providing extendible and/or pivotal connection(s) between the ball structure and the leverage unit and/or between the femur arm/rod and the other structure of the tool, for example, with said extendible and/or pivotal connection(s) preferably being fixable prior to surgery to make the connections non-moving during surgery.

The femur adjustment tool may be made of stainless steel or other strong material(s) suitable for sterilization and use in surgery. Preferably the fulcrum member is rigid and non-compressible, and the lever unit and femur arm/rod are rigid and/or substantially rigid, so that upon firm and consistent/controlled application of force to the handle end of the femur adjustment tool, firm and consistent/controlled leverage is applied to the femur.

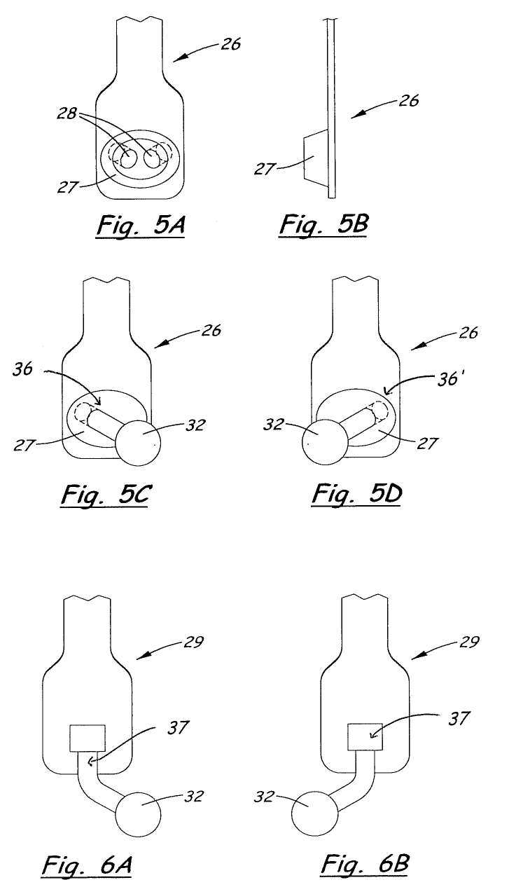

Referring specifically to FIGS. 1-3, 4A-C, 5A-D, 6A and B, 13A and B, 14A and B, 15, 16 and 19 there are shown multiple, but not the only, embodiments of the invented femur adjustment tool, specifically tool 10, tool 20, and tool end portion 25 (FIGS. 13A and B, and 14A and b), tool end portion 26 (FIGS. 5A-D) and tool end portion 29 (FIGS. 6A and B). The preferred fulcrum member is a generally ball-shaped member 32, which may be various ball-like, spherical, or part-spherical shapes, but which is most preferably a “trial head” selected according to conventional technique by the surgeon when selecting the ball-portion of the prosthetic hip-joint to be attached to the femur as part of the hip reconstruction. That is, the fulcrum member is preferably a trial head ball that is preferably close in diameter to the inner surface of the patient's hip socket. Various bases, shafts, or other connections may be used, for example but not limited to the example connections 34, 35, 36, 36′, 37, 39 (see, particularly, FIGS. 1, 4A, 5C and D, 6A and B, and 13B, respectively). Said connections optionally may be adapted to comprise adjustability features, for example, the ability to lengthen by rotating in a turnbuckle fashion or by other means, allowing the portion of the tool that contacts the femur (for example, rod or “arm” 42, 44 to better reach to the femoral neck for the function of moving the femoral neck away from, and elevating it relative to, the acetabulum. Further, said connections may optionally comprise adjustability features for shifting the location of the ball member to the right or left, or up or down, on the endplate of the tool, as will be discussed below in more detail.

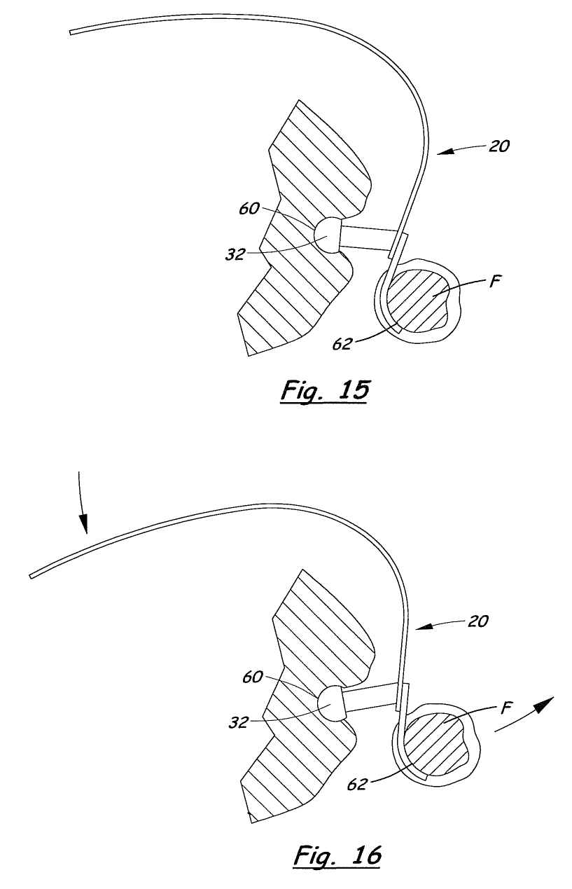

With the femur adjustment tool is securely positioned with the ball pivoting in the acetabulum and the rod 42, 44 slid behind (posterior to) the femur neck, leveraging the tool 10, 20 causes the rod 42, 44 to move the proximal femur upwardly and laterally. This is due to the locations of the ball 32, the handle 50, and the rod 42, 44, relative to each other, and in part to the curved shape of the rod. One may see to best advantage in FIGS. 2, 15 and 16, that downward pressure on the handle 50, with the ball 32 pivoting (“rolling”) in the hip joint acetabulum (“socket” or “cup” 60), will cause the rod 42 to rotate (upwards and to the left in FIG. 2, and upwards and to the right in FIGS. 15 and 16). In addition to, or instead of, the optional adjustability of the ball connection to allow the rod to better reach the femur (discussed above), the rod 42, 44 may optionally be adjustable (extendible and/or retractable) in length and/or have an adjustable connection 46 to the handle 50 (at or near handle portion 51) so that the rod 42, 44 is better adapted to reach the appropriate surface(s) of the femur of each particular patient. Alternatively, various sizes and lengths of tools and/or tool portions may be provided for various patients; for example, the rod 42, 44 is preferably available in a selection of shapes and sizes to allow accommodation of individual variation in patients' builds and dimension. The rod 42, 44 material may be stainless steel, for example, or high grade strong plastic, or other sterilizable materials.

In addition to optional adjustability of the length of the connection between the ball and the main body of the tool and/or the extent to which the rod extends from the main body of the tool, the location of connection of the ball and/or rod may be lockably adjusted and/or lockably changed to allow adjustment and/or fine-tuning of the tool. For example, the tool 10 shown to best advantage in FIGS. 1-3, includes a fulcrum member (ball-shaped member 32) that has is secured and fixed to the main body of the tool in one place and is not moveable to other locations on the tool. The femur rod 42 of tool 10, however, is moveable to either of the right or left connections 46, for adaptation to better fit each of a human's hips. While the rod is connected to the handle 50 by methods that fix it immovably relative to the handle 50 during use, the rod 42 may be removed, switched to the alternate connection 46, and re-fixed, prior to use in surgery. This way, such an adjustable and lockable tool may be used for both a right hip and a left hip. For example, one may see, in FIGS. 1-3, two connection 46 structures for rod 42, so that the rod may be moved to either side of the centerline of the tool for left and also right hip surgery. In left hip surgery, for example, it will be easier to lift and lateralize the femur if the rod 42 is slightly distal (nearer the knee) relative to the ball when positioned in the patient. The rod 42 may be removed and switched to the other of the connections 46, prior to surgery for a right hip arthroplasty and/or as the surgeon sees fit.

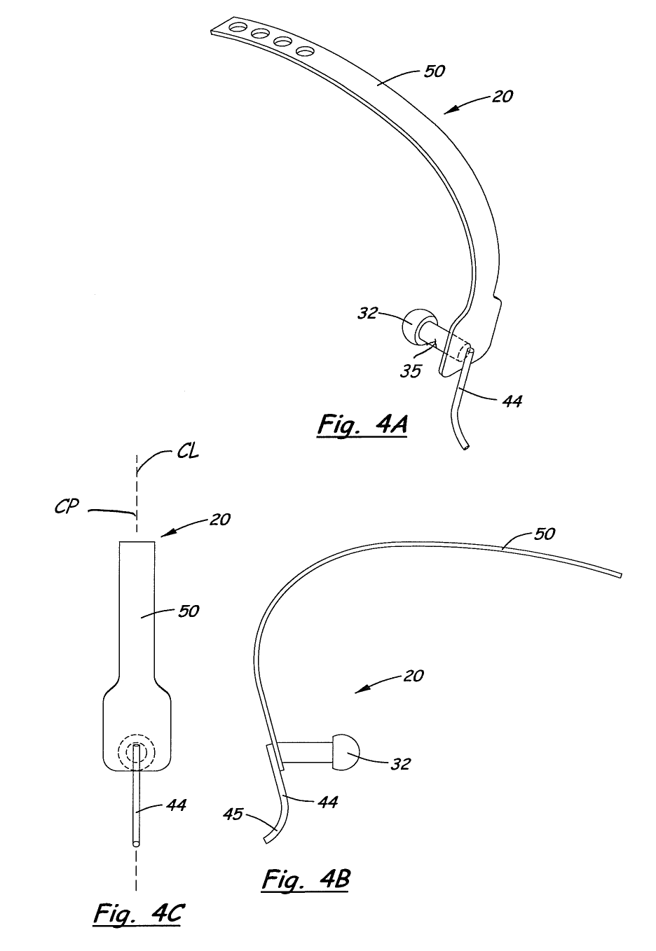

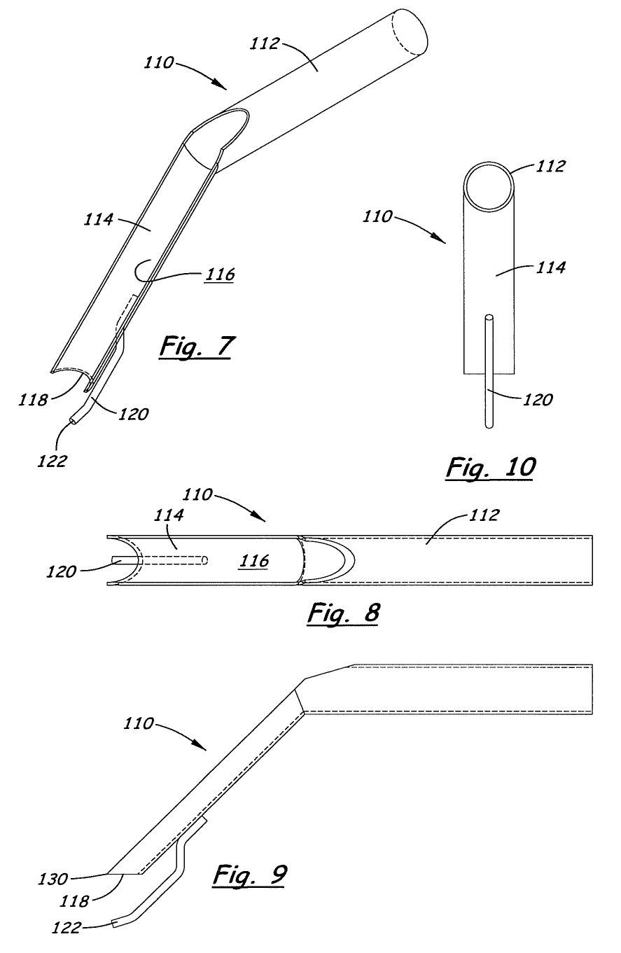

FIGS. 4A-C illustrate an alternative embodiment, femur adjustment tool 20, that comprises both the ball-structure 32 (connection 35) and the femur rod 44 fixed to the tool on the centerline of the tool main body. This tool is used to portray the preferred methods of use of the femur adjustment tool, in FIGS. 15, 16, and 19, for simplicity, and to illustrate the option of limited or no adjustability for some embodiments of the tool. In most instances, however, the preferred methods will include the femur rod being offset laterally relative to the ball to better reach and leverage the neck of the femur. Therefore, while the femur adjustment tool may have fixed and non-adjustable connections for the ball and/or the femur tool in some embodiments, said fixed and non-adjustable connections will typically place the ball and the femur rod offset laterally relative to each other. See, for example, see FIGS. 8-11 of Provisional Application Ser. No. 61/083,460, which is incorporated herein. For example, fixed connections may comprise the ball secured and fixed on the centerline of the tool, with the femur rod secured and fixed to the endplate of the tool toward one or the other side of the ball. For example, the femur rod might be secured and fixed, immovably and permanently, to the left of the ball in FIG. 4C for a left-hip tool (because the left-leg femur neck will be offset relative to the acetabulum), and to the right of the ball in FIG. 4C for a right-hip tool (because the right-leg femur neck will be offset relative to the acetabulum in the other direction). Or, for example, fixed connections may comprise the femur rod being secured and fixed on the centerline of the tool, and the ball being secured and fixed to either the left or the right of the ball. In such embodiments, the surgeon would have two tools, a left-hip tool and a right-hip tool, “in his bag” to select for surgery.

Referring specifically to FIGS. 5A-D, 6A and B, and 13A and B, there are shown alternative systems for adjusting the location of the relative location of the ball and the femur rod. FIGS. 5A-D illustrate tool end portion 26 (wherein the femur rod is not shown), wherein a protruding connection system 27 comprises two connection holes 28 into which the shaft of the ball 32 may be fixed. One may see, in FIGS. 5C and D that the connection system 27 and its holes 28 are adapted so that fixing the ball shaft into one of the holes 28 will cause the ball to extend in one direction (to the right in FIG. 5C) beyond the end plate of the end portion of the handle (or curved “main body”), and fixing the ball shaft into the other of the holes 28 will cause the ball to extend in the other direction (to the left in FIG. 5D) beyond the end plate of the end portion of the main body. It may be assumed that the preferred femur rod in this embodiment (not shown in FIGS. 5A-D) would be secured and fixed at the centerline of the end plate on the opposite side of the end plate from the ball.

FIGS. 6A and B illustrate an alternative embodiment, end portion 29, which comprises a single connection system 37 in which a curved/bent ball shaft may be adjustably locked. In the position of FIG. 6A, the ball shaft is locked after being swiveled so that the ball extends to the right, and in the position of FIG. 6B, the ball shaft is locked after being swiveled so that the ball extends to the left. This way, the connection system 37, by set-screw of other lock, may cause the ball member 32 to extend laterally in multiple directions away from the centerline of the tool.

Referring specifically to FIGS. 13A and B, and FIGS. 14A and B, the end portion 25 of another alternative tool is portrayed wherein the ball structure 32 may be attached in multiple positions and the rod 44 is preferably left immovable and non-adjustable on the tool at or near the longitudinal center-plane of the tool 30. It is preferred that two threaded holes or other attachment points be provided side-by-side on the lever unit which are adapted so that the ball can be fixed to each attachment point. In one position, connected at connection 38, the ball extends outward from the endplate 52 of the tool, from nearer one edge of the tool, and preferably about 30 degrees down from horizontal and about 30 degrees to the one side from the centerplane of the tool. In the other position, connected at connection 38′, the ball extends outward from the endplate 52 of the tool, from nearer the opposite edge of the tool, and preferably about 30 degrees “down” (relative to a horizontal plane when the end plate is vertical) and about 30 degrees to the opposite side (relative to the vertical centerplane when the endplate is vertical). This preferred orientation is illustrated to best advantage by the obtuse angles D and O in FIGS. 14A and B. Angle D (for “down”) of the connection axis of the ball to the main plane of endplate 52 (main plane of the endplate being parallel to the paper in FIG. 6, and preferably parallel to its vertical front and rear surfaces) is preferably in the range of 110-130 degrees, more preferably 115-125 degrees, and most preferably 120 degrees. Also, angle O (for “out to the side”, when viewed in the orientation of FIG. 14B) of the connection axis of the ball to the main plane of endplate 52 is in the range of 110-130 degrees, and more preferably 115-125, degrees, and most preferably 120 degrees.

Therefore, rather than adjusting the actual connection of the ball or other fulcrum member to the lever unit main body (handle unit) by lengthening the connecting shaft or screwing the shaft closer toward or farther away from the endplate 52, the ball may be moved to alternative connection points on the endplate prior to use. Still, in alternative embodiments, adjustable connections may include one or more of the following, for example: for lengthening or shortening the connection (lengthening or shortening the distance from the surface of the ball to the lever bar endplate; and/or pivoting/swiveling the ball in one or more directions relative to the lever bar; and/or providing multiple differently located attachment structure on the lever unit, wherein each of the attachment structures will preferably result in the ball structure extending out from the lever unit at different locations relative to the center line/center plane of the tool and extending out from the lever emit angled toward one side edge or the other. Each of these adjustable connections, or alternative attachment points, should be lockable or latchable or immovable, so that, during surgery, the femur adjustment tool's moveable/adjustable portions (preferably moveable during adjustment only) are completely fixed and immoveable during surgery and not movable relative to each other during use of the tool.

Various other connection systems, other than those shown, may be used for placement and/or fine-tuning of the ball structure relative to the femur-contacting structure. For example, threaded holes or other receivers, set-screws, clamps, pins, or other locks/latches may be used for adjustable connections. Conventional methods for non-adjustable connections may be used, for example, integral formation and/or molding, welding, screws or other fasteners, may be used.

The preferred femur-contact members, for example, rod 42, 44, are curved and/or bent in shape. For example, in FIGS. 1-3, the femur rod extends from the end/edge of the endplate of the lever bar (main body/handle) so that the longitudinal axis of the portion of the rod 42 near the endplate is generally parallel to the longitudinal axis of the endplate 52, but with the outer end of the femur rod 42 curving slightly away from the lever bar endplate 52 in a direction opposite from the fulcrum member, and also curving or bent laterally relative to the endplate 52. For example, in FIGS. 4A-C, rod 44 does not curve/bend laterally, and instead lies on or substantially on the centerplane CP of the tool. Rod 44 curves away from the centerline of the tool, and away from ball 32, to form curved end 45. One may see the centerplane CP schematically portrayed in FIG. 4C (wherein it is a vertical plane extending into the paper of FIG. 4C) and one may further understand centerline CP as extending through the longitudinal axis of the tool and being parallel to the plane of the paper, in FIG. 4B. One may note that the longitudinal axis (also “centerline”) of the tool will curve along the main body (handle) of the line generally equidistant from the side edges of the main body, and is also illustrated as line CL in FIGS. 1 and 4C.

Curving, bending, lockable pivoting or swiveling, or other shapes or adjustments that move the end of the rod 42 away from the centerplane of the tool preferably will not be so substantial as to interfere with leverage control and effectiveness by substantially changing the longitudinal axis shape/configuration of the lever unit. Further, curving, bending, lockable pivoting or swiveling, or other shapes of adjustments of the ball structure 32 away from the centerplane of the tool preferably will not be so substantial as to interfere with leverage control and effectiveness by substantially changing the longitudinal axis shape/configuration of the lever unit. In other words, offset of the femur rod and/or ball is typically desired for optimum reaching of the acetabulum and/or femur neck and consequent optimum operation, but should not be so extreme that it makes appropriate and controlled leverage difficult or impossible to accomplish.

### Tissue Protector and Broach Guide Tool

FIGS. 7-11 illustrate the preferred embodiment of the tissue protector and broach guide tool 110 (“TPG”). TPG tool 110 comprises a handle end 112, a protector end (or “protector portion”) with end 118 and with curved guide surface 116, and an arm or hook 120 attached or connected preferably to the rear surface of protector portion 114 and curving/bent to extend outward and then generally toward (but not touching) end 118. The handle end 112 is preferably a generally straight member at an acute angle in the range of 130-160 degrees (more preferably 150-160 degrees) relative to the protector portion 114. The protector portion 114 may also be said to be a generally straight member, with a curved upper surface (surface 116). The relative lengths of the handle end 112 and the protector portion will be determined by the size and length of the broaching tool and the leverage that is desired against the tissue around the incision.

The TPG tool 110 may be used in the general location of conventional retractors that have been used in the past to retract skin and other soft tissue around and in the incision during hip arthroplasty. See, for example, conventional retractor R1 in FIGS. 7-10 of Provisional Application 61/155,148, incorporated herein, which conventional retractor is used for soft tissue retraction and/control but does not have the features and benefits of the invented TPG. The TPG tool 110 has an arm or hook 120 that “hooks under” the femur to assist in lifting and lateralizing the femur and/or in indexing and securely locating and stabilizing the TPG tool for improved stability and safety of the broaching step. The TPG tool 110 further has structure, the protection portion 114, and particularly the region of portion 114 that is near end 118, that cooperates with the hook 120 to further stabilize the tool, because the hook 120 extends around one surface of the femur top (around the posterior of the femur) and the protective portion 114 extends along the opposite (anterior) surface of the femur.

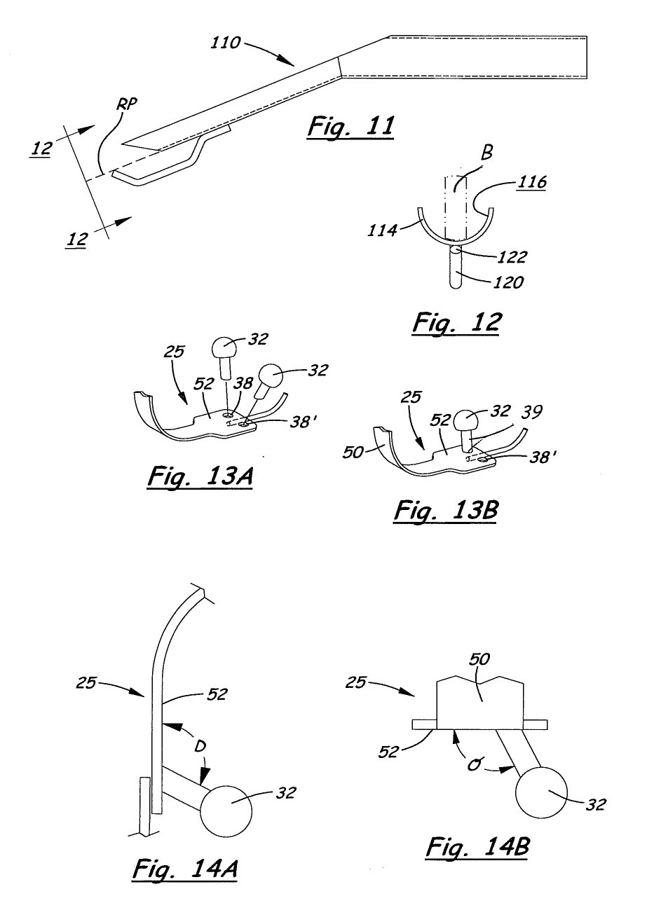

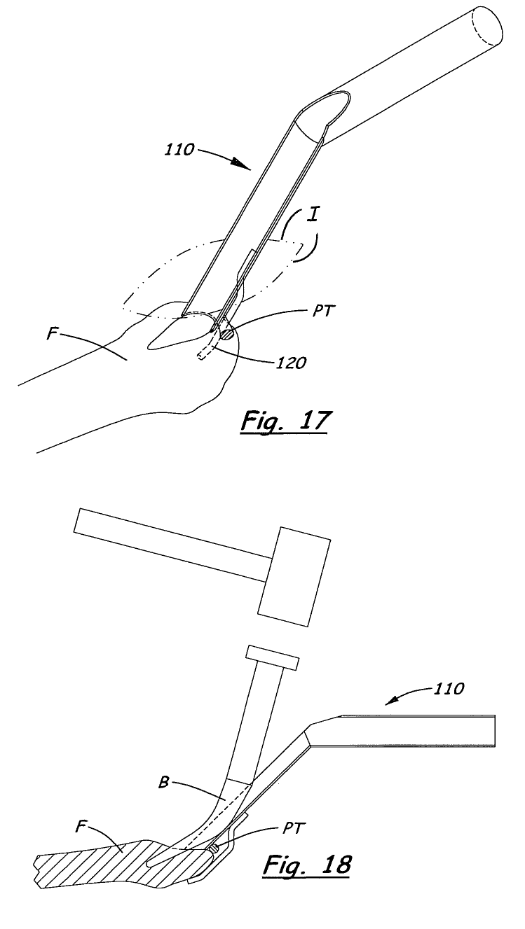

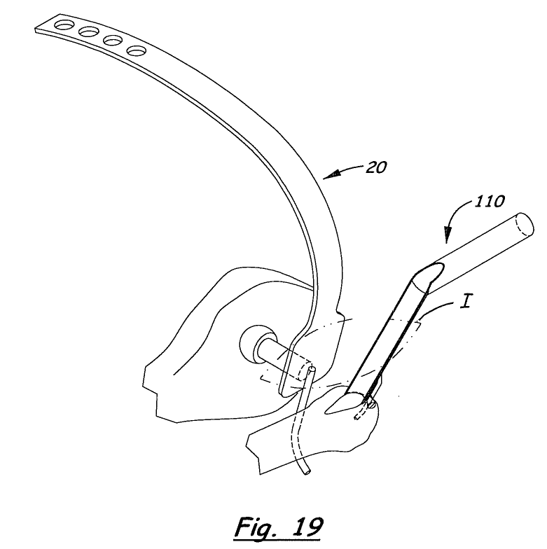

Further, the protection portion 114, and the significant side-to-side curvature of the guide surface 116 of protection portion 114 of the tool, protect the surrounding tissue and guide the broaching tool (B in FIG. 18) to prevent it from slipping to one side of the other and impacting/cutting surrounding tissue. This is especially important for protecting the piriformis tendon (PT in FIGS. 17 and 18), which is located near the femur end and, given the structure and method of using the preferred TPG, will be sheltered and shielded between the protection portion 114 (especially the region near end 118) and the hook 120. Given this location of the tendon, and given the significant curvature of the tool main body, the broaching tool B will not slide off of the tool on its way toward the femur for cutting/drilling, and will not harm the tendon. One may note in FIG. 18, that the broaching tool B must “turn the corner” as it is hammered toward the tool (downward in FIG. 18) because it must actually cut/drill toward the left in FIG. 18 into the femur. The curved guide surface 116 of the tool helps greatly in this movement and stable drilling. The guide surface 116 is preferably a half-cylindrical or generally half-cylindrical, as shown in FIG. 12, but may be other curved, recessed, and/or generally concave surface shapes. As also shown in FIGS. 11 and 12, the extremity (end 122) of hook 120 preferably reaches to, or nearly to, the rear plane RP of the tool protection portion 114, and, in view of the hook 120 preferably being centered along the length of the tool, the end 122 preferably extends to, or near to, the longitudinal centerline of the tough of the curved guide surface (also represented by RP in FIG. 11).

Handle 112 of the preferred embodiment is tubular, but may be other shapes as the main function of handle 112 is for gripping by the user (typically, a surgeon's assistant) and for application of leverage and/or controlling force. All of the TPG tool 110 should be made of durable, rigid, and sterilizable material(s).

In use, the femur adjustment tool (such as tool 20, in FIGS. 15 and 16) is partially inserted into the incision I, with the ball structure (such as ball 32) in the acetabulum against the “cup” or “socket” surface 60. The femur-contacting structure (such as rod 42, 44) is hooked around the femur F to contact a posterior surface 62 of the femur neck. Then, force is applied to the outer end of the handle 50, tool 20 pivots on its fulcrum structure (ball 32), and the femur-contacting structure lifts and lateralized the femur (upwards and to the right in FIG. 16).

In use, the tissue protector and broach guide tool (TPG) (such as tool 110 in FIGS. 17 and 18) is partially inserted into the incision, with the end 118 resting against an anterior surface of the femur near the end of the femur that has been disconnected from the acetabulum (hip cup or socket). During this insertion, the hook 120 is placed around the posterior end of the femur, as shown in dashed lines in FIG. 17, so that the hook 120 extends around (to the right of, in FIGS. 17 and 18) the piriformis tendon PT and/or other tissue near the tendon. This way, the tendon PT is received in a space between the rear surface of the protection portion 114 and the hook 120, and is shielded by portion 114. The cooperation of portion 114 and the hook 120, in view of pressure applied to the handle by a user, stabilized the tool 110 on the femur, and makes it unlikely to slide or fall off the femur during the broaching step. Broaching, or other drilling or cutting into generally the longitudinal axis of the femur, is done generally but not necessarily identically as shown in FIG. 18. Impact or other force is applied to the broaching tool B and it is typically not an easy task to “turn the corner” with the broach/drill tool to transmit a generally downward force (downward in FIG. 18 and typically generally downward on the operating table) into a generally horizontal force (in FIG. 18 and on the operating table) to drive the broaching/drilling tool along the longitudinal axis of the femur. Therefore, the TPG tool 110 is very beneficial in that it is adapted to provide a curved guide surface that is stabilized against the femur itself and that, at the same time as it guides the broaching/drilling tool, it shields the important tendon/soft tissue at or near the femur end.

### Tools in Use in Combination

FIG. 19 illustrates an embodiment of the femur adjustment tool (tool 20, for simplicity) being used in combination with an embodiment of the TPG tool (tool 110, for example). One may see that the centerplane of tool 20 is generally 90 degrees to the centerplane of tool 110, as the endplate of tool 20 is inserted into the incision about 90 degrees from insertion of the protection portion 114 and end 118 of tool 110. The handles 50 and 112 extending away from the incision at about 90 degrees to each other and will typically be operated by two different people. The broaching tool is not shown in FIG. 19, but, from FIG. 18, it will be understood that prior to and during use of the broaching tool, the user of tool 20 will be leveraging the femur into its preferred lifted and lateralized position, and he user of tool 110 will be pressing tool 110 against the femur (and preferably slightly rearward-pivoting the tool 110). Said rearward-pivoting of tool 110 may be done by using pivoting the tool 110 against the tissue around the incision (at I in FIG. 19) to leverage against the tissue, which may further lift and/or at least stabilize the position of the femur and/or may retract and/or control said tissue (at I) around the incision. For the orthopedic surgeon, in doing a small-incision anterior approach for hip replacement surgery that does not cut muscle, exposure of the proximal femur for broaching in preparation to fit the femoral component is difficult. Moving the proximal femur away from the acetabulum and pelvis is difficult, and accurately and safely broaching the femur may be difficult depending on how well the femur is moved away from the acetabulum and pelvis. However, by using the preferred combination of femur adjustment tool and TPG tool, more surgeries may be done effectively, efficiently, safely, and with minimal invasion into the patient's body, which translates into better results and faster recovery.

### Bone Clamp

The preferred tool for clamping a bone portion, for example in knee arthroplasty or other orthopedic surgery, is portrayed in FIGS. 20-22. The preferred embodiment is used for a bone piece that has been cut from a bone, wherein the clamp allows improved holding and/or manipulation of the bone portion in whatever subsequent steps are conducted during the surgery. In view of the trends to reduce incision size and make surgery less invasion, the invented clamp can help make easier the steps of manipulating, freeing, and removing a bone portion, which tends to allow a safer and less aggressive surgical procedure overall. In preferred embodiments, the clamp is adapted for use in operations that require a generally transverse cut across a bone, for example, a proximal tibial cut during a reconstructive/replacement knee operation. See, for example, the preferred methods of using the clamping device in knee arthroplasty, illustrated in FIGS. 23-31. The clamp allows improved control of the bone portion, improved separation of the bone portion from soft tissue attached to said portion, and/or improved removal of the bone portion from the patient's body.

The preferred embodiment of the invented clamp comprises two members pivotally connected together at a pivot axis, which is part-way along the length of the two members, so that the clamping ends (first end of each of the two members) may be moved together or apart, into various clamping positions, by manipulation of the handles at the gripping ends (second end of each of the two members). A latch is preferably provided to retain the clamp in any of the preferred clamping positions so that the user may at least temporarily remove his/her hands from the clamp, or may be more free to manipulate the clamp and the bone piece with less clamping pressure from the human hand, while leaving the bone portion clamped.

One of said first ends preferably comprises a broad, thin, flat plate (hereafter “base-plate”) that is preferably shaped and sized to generally match the freshly-cut bone surface. The other of said first ends is preferably a smaller, narrower arm (hereafter “jaw-arm”) with gripping teeth or other gripping protrusions or surface features. With this combination of clamping structures, the clamping structures may be inserted into the incision, by sliding the base-plate between the bone and the bone portion into the narrow space that has been created by cutting the bone, and by sliding the jaw-arm along/across the opposing surface of the bone portion. Then, upon tightening the clamp and preferably latching it, the surgeon or his/her assistant has captured the bone portion between the clamping structures, with the base-plate providing firm abutment against the entire or substantially the entire cut surface of the bone portion and the jaw-arm forcing the bone portion against the base-plate. This way, control and leverage applied to the bone portion are improved or optimized, and the manipulation of the handles of the clamp allow accurate and sure separation of bone portion and soft tissue and/or accurate and sure removal of the bone portion from the incision. This improved leverage and control allows procedures that feature less aggressive techniques for both the bone cut and the soft tissue resection.

A particularly effective application for the preferred clamp is knee surgery, wherein a proximal tibial cut is made to remove the joint surface piece of the tibia. As the cut ideally is made carefully and non-aggressively in order to minimize damage to the surrounding soft tissue, the resulting joint surface bone piece still has soft tissue attached to it. This residual soft tissue should be detached in a controlled and careful manner before removal of the joint surface piece, and the clamp assists in this task.

Referring to FIGS. 20-31, there is shown one, but not the only, embodiment of the invented tool, which is a clamp device preferably used in clamping/grasping a piece of bone during an operation. The preferred clamp 210 comprises two pivotal members 212, 214, which pivot relative to each other on an axis at a pivot point 216 along the length of the two members 212, 214 between the ends of the members. The pivotal members 212, 214 are generally side-by-side and generally of the same length. The first ends (base-plate 220, and jaw arm 222) of the members 212, 214 serve as the clamping ends and the opposing, second ends of the members 212, 214 serve as the hand-grip ends 215, 217. The pivotal members 212, 214 are each rigid and cross over each other at the pivot 216, so that squeezing the hand-grip ends together forces the clamping ends together. The pivotal movement is preferably continuous within a range of about 20-45 degrees of total movement of the clamping ends relative to each other, and the latch system may be continuous or incremental. In the preferred embodiment, the latch system 218 is incremental, using a notched, ratchet-type latch member 219 so that the user may latch the clamp 210 into many, but not infinite, positions of clamping.

One of the clamping ends is a relatively large plate or base that is substantially wider PW and deeper PD than the other of said clamping ends. In the preferred embodiment, this relatively large clamping end is base-plate 220, which is a generally oval, smooth-edged plate adapted to be the general size and shape of the surface of the bone and bone portion at the cut through the bone. The base-plate 220 is sized in thickness to be easily-slidable into the space (kerf K) formed by the cutting of the bone, for example 1-2 mm, or preferably about 1.5 mm. Also, the base-plate 220 is sized (in diameter, width PW and depth PD) to fill all or substantially all of the transverse dimension of the kerf but preferably not to extend out beyond the perimeter of the kerf, that is, preferably not to extend out beyond the perimeter of the cut bone surfaces. This way, the base-plate 220 may be slid into the kerf so that the base-plate is against (contacting or very near) all of the bone and bone portion surfaces that have been cut apart, but so that the base-plate 220 does not protrude transversely to poke or otherwise interfere or damage residual or other surrounding soft tissue. The base-plate may alternatively be shapes other than oval, such as circular, generally triangular, generally figure-shape, or other shapes to fit the transverse cross-section of various bones.

Thus, in the knee joint replacement operation, of which a few steps are shown in FIGS. 23-31, the base-plate 220 is preferably sized to generally match the upper surface 200′ of the bone 200, and, in doing so, the base-plate 220 will also match the lower surface of the top portion of bone (bone-piece 201). This matching of size provides the doctor the maximum safe leverage against the bone surfaces without making the base-plate unnecessarily large and difficult to insert into the kerf, and/or difficult to remove from the intentionally-small incision. The base-plate 220, therefore, matches or is only slightly smaller than the diameter (or other transverse dimension) of the largest bone piece to be removed from the patient, as portrayed in FIG. 30, for example.

Base-plate 220 is preferably removable from the clamp 210 and interchangeable with base-plates of other sizes, and optionally other shapes. This way, the doctor may select a base-plate 220 that substantially matches the surface area and surface shape of the bone where the bone has been cut (as described above, the bone surfaces facing the kerf, formed at the time of the cut). For example, in a knee replacement operation, a set of base-plates 220 may be provided from which the doctor will chose the desired base-plate. The set of base-plates will typically be based on (similar to) the set/sizes of tibial components (metal prosthesis) from which the doctor chooses a single component that will become the patient's artificial tibial “cup.” Thus, when the doctor picks the size of the tibial component, he/she may be, in effect, also selecting the size of base-plate 220.

The base-plate 220 may be fixed to the pivotal member 212 by various means, such as a set-screw-style system 231 screw or other fastener that will allow safe attachment, safe detachment after use, and proper cleaning and sterilization of the clamp parts and/or the entire clamp. As an alternative to the base-plate 220 being removable and interchangeable, multiple clamps with various sizes of base-plate may be provided.

The other of the clamping ends is preferably a smaller, narrower arm (hereafter “jaw-arm 222”) with gripping teeth 232 or other gripping surface facing the base-plate 220. Jaw-arm 222 opposes base-plate 220, so that the bone portion 201 is grasped and clamped between them, with a relatively broad supporting base surface below the bone portion 201 and the gripping, narrow surface above the bone portion 201. The jaw-arm 222 width JW is sized and shaped to be narrow (small) in the direction parallel to the width PW of the base-plate 220, for example, equal to or less than 33% of the width PW of the base-plate 220, and, more preferably, 15-25% of the width PW. The jaw-arm 222 depth JD may be approximately equal to, or shorter than, the depth PD of the base-plate 222, but, more preferably is in the range of 60-90%, or most preferably in the range of 75-80%, of PD. This allows the jaw-arm 222 to be narrow enough to fit easily into the intercondylar notch N of the femur F, which is closely adjacent the bone portion 201 in this operation, but long enough to supply sufficient and well-directed clamping force against the bone portion 201 in view of the clamping force applied by the base-plate.

The base-plate 220 is preferably smooth and substantially or entirely flat, so that the base-plate 220 may be easily inserted into the kerf K, which may only be approximately 1.5 mm between the bone 200 and the bone portion 201, for example, but which will typically be flat and planar due to being cut with a planar blade. Once inserted, there is a large amount of contact area between the base-plate 20 and the bone 200 and/or bone portion 201 surfaces. The bone portion 201, therefore, may be firmly and securely clamped by the clamp 210, with the base-plate 220 acting as support and base and the jaw-arm 222 acting as a grip arm. The clamping force and/or manipulation force (holding, tilting, lifting, pulling, or otherwise manipulating the clamp and the bone portion 201) will tend, therefore, to be a steady, sure and predictable procedure wherein the bone portion 201 will not tend to slip, slide, wobble, or crack in the clamp 210 during any step of the operation. Especially, the bone portion 201 will be held and/or manipulated in a stable and sure manner during the doctor's removal of the soft tissue from the bone portion 201. With this clamp 210 firmly grasping and latched onto the bone portion 201, the doctor may more conveniently, accurately, and safely work with a tool in another hand to “pop” off the residual tissue from the bone portion 201.

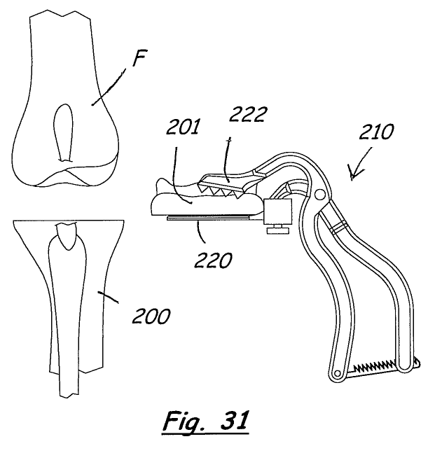

In use, preferred steps may include, but not necessarily be limited to, those shown in FIGS. 23-31, wherein FIGS. 23-26 shown anterior views and FIGS. 27-31 show lateral views of a right knee. Note that a lateral ligament is shown as cut/removed in FIGS. 29 and 31, for ease of viewing the procedures, but that, in the preferred minimally-invasive techniques, cutting and damage of ligament is minimized or avoided altogether.

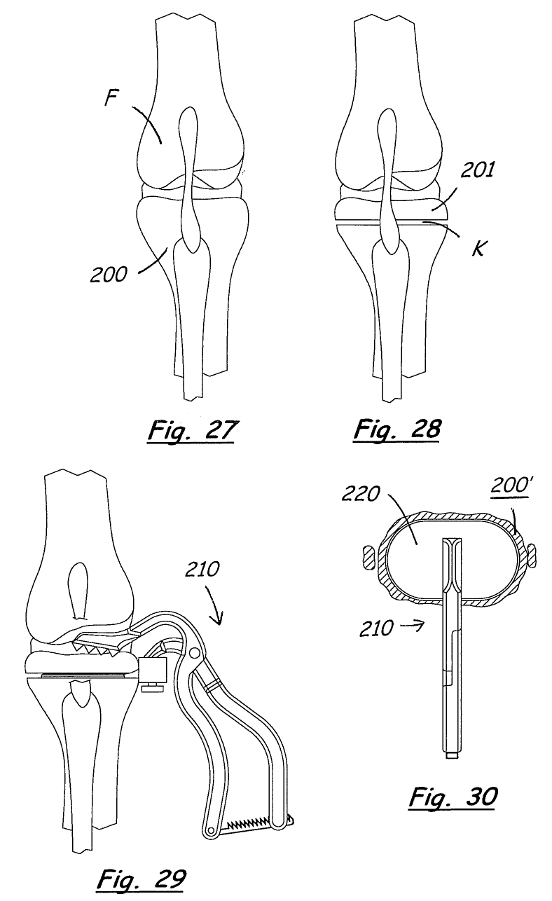

A small incision is made (not shown in FIGS. 20 -31) to provide access to the knee structure, including bone 200 (FIGS. 23 and 27). The cut (kerf K) is made, as in FIGS. 24 and 28 (saw/cutting tool now shown). The preferred bone clamp tool 210 is inserted, so that the clumping ends are slid and/or otherwise manipulated into their respective preferred locations relative to the bone and soft tissue. Specifically, the base-plate 220 slides and/or is otherwise manipulated into the kerf K, and the jaw-arm 222 slides and/or is otherwise manipulated into notch N (FIGS. 25 and 29). The doctor or assistant then tightens the clamp, by squeezing the handle ends 215, 217 together, and preferably latches it using latch 219 (FIG. 29). Thus, the surgeon or his/her assistant has captured the bone portion 201 between the jaws, as illustrated in FIGS. 25 and 29, with the base-plate 220 providing firm abutment against the entire or substantially the entire cut surface of the bone portion 201 (FIG. 30). This way, control and leverage applied to the bone portion are improved or optimized, and manipulation of the handles of the clamp allow accurate and sure separation of bone portion and soft tissue, and removal of the bone portion 201 from the surrounding knee structure and tissue. See FIG. 31, representing the user removing the bone portion 201 toward the right and out of the incision. See, also, the resulting absence of the tool 210 and the bone portion 201 in FIG. 26. The improved leverage and control, provided by the structure and operation of clamp 210, allow efficient and accurate removal of the bone portion from the bone tissue and incision even in the case of a small incision, minimally-invasive bone sawing, and minimally-invasive soft tissue resection and retraction. Therefore, less-aggressive techniques for both the bone cut and the soft tissue resection are made possible by the preferred embodiments of the bone clamp.

Preferred embodiments of the clamp may be simple, effective, and easily-sterilized. The clamp, in its simple embodiments, may consist essentially of, or consist only of, two rigid pivoting members, with one of the pivoting members comprising a broad plate end opposite a narrow gripping end. The handles are preferably curved to extend approximately 90-160 degrees (and preferably 110-140, and more preferably about 135 degrees) from the from the plane of the broad plate for excellent handling and control of the clamp relative to the patient's body and incision, during the clamping, soft tissue removal, and subsequent steps.

It should be noted that the invented femur adjustment tool, the invented tissue protector and guide tool, and the invented bone clamp may be used in operations other than hip, knee, and leg operations, and that the invention may comprise other methods of using the invented tools.

Other embodiments of the invention will be apparent to one of skill in the art after reading this disclosure and viewing the drawings. Although this invention is described herein and in the drawings with reference to particular means, materials and embodiments, it is to be understood that the invention is not limited to these disclosed particulars, but extends instead to all equivalents within the broad scope of the following claims.

1. A tool system for hip arthroplasty comprising:
a femur adjustment tool comprising:
a main body with a first end comprising a handle and a second end comprising an endplate;
a ball structure connected to said endplate on a rear side of the endplate and a femur-contact arm connected to said endplate on a front side of the endplate;
the ball structure and femur-contact arm positioned on the endplate so that the ball structure is adapted to pivot against an acetabulum surface of a hip joint and the femur-contact arm is adapted to reach around a posterior side of the femur, so that pushing on the handle pivots the femur adjustment tool against the acetabulum so that the femur-contact arm lifts and laterializes the femur relative to an incision.

2. The tool system as in claim 1, wherein said ball structure is adjustable to, and lockable in, multiple connection points on said endplate.

3. The tool system as in claim 1, wherein said femur-contact arm is adjustable to, and lockable in, multiple connection points on said endplate.

4. The tool system as in claim 1, further comprising a tissue protector and guide tool located about 90 degrees from said femur adjustment tool and comprising a protector portion having a concave surface for guiding a broach tool and comprising a hook extending from a rear surface of the protector portion to near an end-extremity of said protector portion but spaced from said protector portion so that a gap exists between the protection portion and an outer end of the hook, wherein said gap is adapted for receiving a tendon when the tissue protector and guide tool is rested on a proximal end of a femur.

5. The tool system as in claim 4, wherein said tissue protector and guide tool has a handle portion that is at an angle of 130-160 degrees relative to the protector portion for being leveraged against tissue at the incision.

6. The tool system as in claim 4, wherein said ball structure is moveable and lockable to a position wherein the ball structure extends to a right side of the endplate and is moveable and lockable to a position wherein the ball structure extends to a left side of the endplate.

7. The tool system as in claim 1, wherein said femur adjustment tool consists only of a curved plate that is the main body, one ball structure moveable and lockable in multiple positions on an end of the curved plate, and an elongated rod fixed to the curved plate that is the femur-contact arm.

8. The tool system as in claim 1, wherein said femur adjustment tool consists only of a curved plate that is the main body, one ball structure that is fixed and moveable on an end of the curved plate, and an elongated rod that is moveable and lockable in multiple positions on the curved plate and that is the femur-contact arm.

9. A tool system for hip arthroplasty comprising:
a tissue protector and guide tool comprising a handle end and an opposite end that is a protector portion having an elongated concave surface for guiding a broach tool, said tissue protector and guide tool comprising a hook extending from a rear surface of the protector portion to near an end-extremity of said protector portion but spaced from said protector portion so that a gap exists between the protection portion and an outer end of the hook, wherein an end-extremity of the protector portion is adapted for placement on a proximal end of a femur and wherein said gap is adapted for receiving a tendon when the end-extremity of the protector portion is rested on a proximal end of a femur.

10. The tool system as in claim 9, wherein said elongated concave surface is generally semi-cylindrical.

11. The tool system as in claim 9, wherein said handle and the protector portion are at an angle of 130-160 degrees relative to each other.

12. The tool system as in claim 9, further comprising a femur adjustment tool that comprises a generally spherical fulcrum member adapted for placement in a hip socket for pivoting against the hip socket acetabulum surface, the femur adjustment tool further having a leverage handle and a femur-contact extension for lifting and lateralizing the femur, wherein said handle of the tissue protector and guide tool and said handle of the femur adjustment tool extend generally 90 degrees relative to each other when installed in a hip arthroplasty incision.

13. A hip surgery method comprising:
providing a femur adjustment tool comprising:
a main body with a first end comprising a handle and a second end comprising an endplate;
a ball structure connected to said endplate on a rear side of the endplate and a femur-contact arm connected to said endplate on a front side of the endplate;
placing the femur adjustment tool in a hip arthroplasty incision so that the ball structure contacts and pivots against a hip socket acetabulum surface and so that the femur-contact arm reaches around a posterior side of the femur; and
pushing on the handle to pivot the femur adjustment tool against the acetabulum, with the ball structure being the fulcrum, so that the femur-contact arm lifts and laterializes the femur relative to the incision.

14. The method of claim 13, further comprising:
providing a tissue protector and guide tool in said incision, the tissue protector and guide tool comprising a protector portion having a concave surface and an end extremity, and a hook extending from a rear surface of the protector portion but spaced from said protector portion so that a gap exists between the protection portion and an outer end of the hook;
positioning the end extremity on an anterior surface of the femur so that the hook extends around to a posterior surface of the femur, so that the gap receives a piriformis tendon associated with the femur; and
the method further comprising:
placing a broach tool against said concave surface and using said concave surface to guide the broach tool as the broach tool is impacted toward and into the femur.

15. A bone clamp comprising;
A first arm and a second arm pivotally connected to each other, said first arm having at one end a plate and said second arm having at one end a jaw, wherein said jaw is narrow compared to the width of the plate, and the plate is removable and replacement, so that said plate is selected to generally match size and shape of a transverse surface area of a bone when a cut has been made through said bone.

16. The bone clamp of claim 15, wherein said jaw has a width that is 15-25% of the width of the plate.

17. The bone clamp of claim 15, wherein the jaw has a depth that is 60-90% of the depth of the plate.

18. The bone clamp of claim 15, wherein the plate is 1-2 mm thick.

19. A method of clamping a bone portion cut from a larger bone, the method comprising:
providing a bone clamp comprising;
a first arm and a second arm pivotally connected to each other, said first arm having at one end a plate and said second arm having at one end a jaw, wherein said jaw is narrow compared to the width of the plate, and the plate is removable and replacement, so that said plate is selected to generally match size and shape of a transverse surface area of a bone when a cut has been made through said bone;
the method further comprising sliding the plate between the larger bone and the bone portion and sliding the jaw into the intercondylar notch of the femur; and
clamping the bone portion between the plate and the jaw and removing the bone portion from an incision.
